(19) United States
(12) Patent Application Publication
(10) Pub. No.: US 2017/0362498 A1
(43) Pub. Date: Dec. 21, 2017

(54) USE OF MGDA AS ADDITIVE IN PROCESSES FOR RECOVERING CRUDE OIL AND/OR GAS FROM SUBTERRANEAN FORMATIONS

(71) Applicant: BASF SE, Ludwigshafen (DE)

(72) Inventors: Jennifer JACKSON, Houston, TX (US); Stefan FRENZEL, Mannheim (DE); Oscar LAFUENTE CERDA, Ebersberg (DE)

(21) Appl. No.: 15/536,766

(22) PCT Filed: Dec. 16, 2015
(86) PCT No.: PCT/EP2015/080055

§ 371 Date: Jun. 16, 2017

(30) Foreign Application Priority Data
Dec. 17, 2014 (EP) 14198557.2

(51) Int. Cl. C09K 8/74; E21B 43/25; E21B 41/02; C07C 229/16; E21B 37/06; E21B 37/00; E21B 43/26; E21B 43/16

(52) U.S. Cl. CPC C09K 8/74; E21B 43/26; E21B 43/25; E21B 43/16; C07C 229/16; E21B 37/06; E21B 37/00; E21B 41/02

(57) ABSTRACT

Use of methyl glycine diacetic acid (MGDA) as additive in processes for recovering crude oil and/or gas from subterranean formations, wherein the MGDA is a mixture of L- and D-enantiomers of MGDA or salts thereof, said mixture containing an excess of the respective L-isomer, and the enantiomeric excess (ee) of the L-isomer is in the range of from 10% to 75% Preferably, the process is a processes of acidizing subterranean formations.

The present invention relates to the use of methyl glycine diacetic acid (MGDA) as additive in processes for recovering crude oil and/or gas from subterranean formations, in particular in processes of acidizing, wherein the MGDA is a mixture of L- and D-enantiomers of MGDA or salts thereof, said mixture containing an excess of the respective L-isomer, and the enantiomeric excess (ee) of the L-isomer is in the range of from 10% to 75%.

MGDA may be prepared by reacting iminodiacetonitrile with acetaldehyde and hydrocyanic acid, or alpha-alaninenitrile with formaldehyde and hydrocyanic acid, and alkaline hydrolysis of the methylglycinediacetonitrile (MGDN)

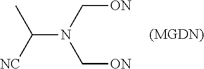

obtained as an intermediate with sodium hydroxide solution to obtain the trisodium salt of MGDA. In order to achieve high MGDA yields and purities, MDGN is isolated as an intermediate and used as a pure substance in the hydrolysis step which follows.

U.S. Pat. No. 7,671,234 discloses an improved process for the saponification of MGDN.

WO 2012/150155 discloses a process for preparing a crystalline L-MGDA tri-alkali metal salt. However, it is tedious to make MGDA and to carefully avoid any racemization. Although it is well possible to synthesize racemic MGDA and to separate off the D-isomer, such a method would result in disposing 50% of the yield or more.

Our older application WO 2015/036324 A1 discloses a mixture of L- and D-enantiomers of methyl glycine diacetic acid (MGDA) or its respective mono-, di or trialkali metal or mono-, di- or triammonium salts, said mixture containing predominantly the respective L-isomer with an enantiomeric excess (ee) in the range of from 10 to 75%. It furthermore discloses a method of manufacturing such mixtures and the use of such for the manufacture of laundry detergent compositions and of detergent compositions for cleaners. The application does not disclose the use of such mixtures of L- and D-enantiomers of MGDA as additive in processes for recovering crude oil and/or gas from subterranean formations.

It is well known in the art to use chelating agents such as methyl glycine diacetic acid (MGDA) or their respective salts in various oilfield applications. MGDA is biodegradable and therefore in particular suitable for that purpose.

U.S. Pat No. 5,783,524 discloses a process for complexing alkaline earth and heavy metal ions in petroleum and/or natural gas using MGDA or salts thereof, in particular in course of production or transportation petroleum and/or natural gas.

US 2008/0153718 A1 discloses a method of acidizing or fracture acidizing carbonatic formations using methane sulfonic acid and optionally further acids. The acid may comprise complexing agents, in particular MGDA.

WO 2012/080297 A1 discloses a method of iron control in course of treating subterranean formations, in particular in course of acidizing formations by adding GLDA and/or MGDA to the treatment fluid.

US 2012/0115759 A1 and US 2012/0097392 A1 discloses the addition of complexing agents to treatment fluids for subterranean formations.

US 2012/0202720 A1, US 2014701116710 Al, US 2014/0124205 Al, WO 2013/160332 A1, WO 2013/189842 A1, US 2013/0264060 A1, and WO 2013/189731 A1 disclose the use of MGDA for acidizing and/or fracturing.

US 2014/0120276 A1 and WO 2013/120806 A1 disclose the reduction of corrosion of oilfield equipment which is in contact with treatment fluids by adding MGDA and/or GLDA to the treatment fluid.

WO 2013/160334 A1 discloses a one-step process of removing filter cake using MGDA.

For the application of chemicals in the oilfield not only the chemical or physico-chemical properties of the chemicals themselves are important for economic and technological success but also formulating and handling the chemicals—and/or formulations thereof—for the application is important. It has to be kept in mind that the amounts of chemicals consumed in oilfield applications may be large. In certain oilfield applications it may be necessary to handle a few hundred tons or even a few thousand tons of oilfield chemicals per year on the oilfield. Furthermore, it has to be kept in mind that space for handling oilfield chemicals may be limited. While space may be no problem for land-based oil production, space on off-shore platforms necessarily is limited.

For the oilfield applications such as those mentioned above, the abovementioned chelating agents such as MGDA are usually used as liquid formulations, in particular as aqueous formulations.

Chelating agents such as MGDA may be shipped as solids such as granules or powders to the oilfield. However, such solids need to be dissolved on the oilfield and the necessary equipment such as vessels for dissolving and tanks for storing the solutions need to be available on the oilfield. It goes without saying that dissolving the chelating agents causes additional efforts. Let aside additional efforts and additional equipment, also space necessary for dissolution stations may be a problem on off-shore platforms. Therefore, usually it is helpful to abstain from the use of dissolution stations for dissolving complexing agents, especially on off-shore platforms.

Many users therefore wish to obtain chelating agents in aqueous solutions that are as highly concentrated as possible. The lower the concentration of the requested chelating agent the more water is being shipped. Said water adds to the costs of transportation. Although about 40% by weight solutions of racemic MGDA trisodium salt can be made and stored at room temperature, local or temporarily colder solutions—for instance solutions stored and/or handled in colder environment such as arctic regions- may lead to precipitation of MGDA, as well as nucleating by impurities. Said precipitations may lead to incrustations in pipes and containers, and/or to impurities or inhomogeneity during formulation.

Basically, it is possible to increase the solubility of chelating agents by adding suitable solubilizing agents, such as for instance a solubility enhancing polymer or a surfactant. However such additives inevitably become also components of the formulations used may cause adverse and undesirable effects in the desired oilfield application.

It has further been found that racemic MGDA shows some intolerance against strong bases such as sodium hydroxide. This limits its usefulness in certain oilfield applications in which complexing agents are used together with bases such as alkali-surfactant-flooding or alkali-surfactant-polymer-flooding.

It was an object of the present invention to provide an improved method of using MGDA for oil-field applications, in particular an improved method of using aqueous formulations of MGDA for oilfield applications.

Surprisingly, it has been found that the object can be achieved by using mixtures comprising L- and D-enantiomers of MGDA having an enantiomeric excess of the L-isomer for oilfield applications. Such mixtures of enantiomers having an enantiomeric excess of the L-isomer yield more stable solutions in water than a racemic mixture of the enantiomers and therefore may be formulated in water at higher concentrations. So, handling of MGDA on the oilfield becomes improved by the use of said mixtures having an enantiomeric excess as compared to using racemic mixtures.

Accordingly, the use of methyl glycine diacetic acid (MGDA) as additive in processes for recovering crude oil and/or gas from subterranean formations has been found, wherein the MGDA used is a mixture of L- and D-enantiomers of MGDA or its respective mono-, di or trialkali metal or mono-, di- or triammonium salts, said mixture containing an excess of the respective L-isomer, wherein the enantiomeric excess (ee) of the L-isomer is in the range of from 10% to 75%.

In a preferred embodiment of the invention, the process is an acidizing process.

Specific details of the invention are as follows:

MGDA Mixture to be Used

For the use of MGDA as additive in processes for recovering crude oil and/or gas from subterranean formations according to the present invention mixtures of L- and D-enantiomers of methyl glycine diatetic acid (MGDA) or its respective mono-, di or trialkali metal or mono-, di or triammonium salts are used, said mixture containing an excess of the respective L-isomer, wherein the enantiomeric excess (ee) is in the range of from 10 to 75%, preferably from 12.5 to 60%.

In enantiomeric mixtures, the term “enantiomeric excess (ee)” refers to the excess of all L-isomers present in the mixture compared to all D-isomers. For example, in cases wherein a mixture of the di- and trisodium salt of MGDA is present, ee refers to the sum of the disodium salt and trisodium salt of L-MGDA with respect to the sum of the disodium salt and the trisodium salt of D-MGDA.

The enantiomeric excess can be determined by measuring the polarization (polarimetry) or preferably by chromatography, for example by HPLC with a chiral column, for example with one or more cyclodextrins as immobilized phase or with a ligand exchange (Pirkle-brush) concept chiral stationary phase. Preferred is determination of the ee by HPLC with an immobilized optically active amine such as D-penicillamine in the presence of copper(II) salt.

The term “ammonium salts” as used in the present invention refers to salts with at least one cation that bears a nitrogen atom that is permanently or temporarily quaternized. Examples of cations that bear at least one nitrogen atom that is permanently quaternized include tetrame-thylammonium, tetraethylammonium, dimethyldiethyl ammonium, and n-C₁₀-C₂₀-alkyl trimethyl ammonium. Examples of cations that bear at least one nitrogen atom that is temporarily quaternized include protonated amines and ammonia, such as monomethyl ammonium, dimethyl ammonium, trimethyl ammonium, monoethyl ammonium, diethyl ammonium, triethyl ammonium, n-C₁₀-C₂₀-alkyl dimethyl ammonium 2-hydroxyethylammonium, bis(2-hydroxyethyl) ammonium, tris(2-hydroxyethyl)ammonium, N-methyl 2-hydroxyethyl ammonium, N,N-dimethyl-2-hydroxyethylammonium, and especially NH₄⁺.

In one embodiment of the present invention, the mixtures used are mixtures of L- and D-enantiomers of molecules of general formula (I)

[CH₃—CH(COO)—N(CH₂-COO)₂]M₃₋ₓHₓ (I)

wherein
x is in the range of from zero to 0.5, preferably from zero to 0.25,
M is selected from substituted or non-substituted ammonium, preferably unsubstituted ammonium and potassium and sodium and mixtures thereof, preferably sodium,

said mixture containing an excess of the respective L-isomer, wherein the enantiomeric excess (ee) is in the range of from 10 to 75%, preferably in the range of from 12.5 to 60%.

In one embodiment, trialkali metal salts of MGDA such as the tripotassium salts or trisodium salts, preferably the trisodium salts may be used.

In another embodiment of the invention MGDA may be used as acid, i.e. as MGDAH₃.

In one embodiment of the present invention, the invention relates to mixtures of L- and D-enantiomers of molecules of general formula (II) and their use for for recovering crude oil and/or gas from subterranean formations

[CH₃—CH(COO)—N(CH₂—COO)₂]M_{3-y}H_{y} (II)

wherein
y is in the range of from 0.75 to 2.9, in particular from 1 to 2.5, preferably from 1.5 to 2.5,
M is selected from ammonium, substituted or non-substituted, and potassium and sodium and mixtures thereof, preferably sodium,

said mixture containing an excess of the respective L-isomer, wherein the enantiomeric excess (ee) is in the range of from 10 to 75%, preferably in the range of from 12.5 to 60%.

In one embodiment of the present invention, the mixtures used may contain in the range of from 0.1 to 10% by weight of one or more optically inactive impurities, at least one of the impurities being at least one of the impurities being selected from iminodiacetic acid, formic acid, glycolic acid, propionic acid, acetic acid and their respective alkali metal or mono-, di- or triammonium salts.

In one aspect of the present invention, the mixtures used may contain less than 0.2% by weight of nitrilotriacetic acid (NTA), preferably 0.01 to 0.1% by weight.

In one aspect of the present invention, the mixtures used may contain minor amounts of cations other than alkali metal or ammonium. It is thus possible that minor amounts, such as 0.01 to 5 mol-% of total chelating agent inventive mixture, based on anion, bear alkali earth metal cations such as Mg²⁺ or Ca²⁺, or transition metal ions such as Fe²⁺ or Fe³⁺ cations.

The mixtures described display a very good solubility, especially in water and aqueous alkali metal hydroxide solutions. Such very good solubility can be seen, for example, in a temperature range of from zero ° C. to 40° C., in particular at room temperature and/or at zero and/or +10° C.

In one embodiment of the invention the described mixture of L- and D-enantiomers to be used according to the present invention may be present as aqueous solution containing in the range of from 40 to 60% by weight of said mixture, preferably 45 to 58% by weight, even more preferably 48 to 55% by weight. Such concentrated aqueous solutions may be used to ship MGDA to the oilfield where they may be diluted and/or mixed with further components to obtain suitable formulations for the respective oilfield use. Such concentrated aqueous solutions do not show amounts of precipitation or crystallization on addition of seed crystals or mechanical stress and do not exhibit any visible turbidity.

In one embodiment of the present invention, the concentrated aqueous solutions are free from surfactants. “Free from surfactants” shall mean, in the context of the present invention, that the total contents of surfactants is 0.1% by weight or less, referring to the amount of inventive mixture. In a preferred embodiment, the term “free from surfactants” shall encompass a concentration in the range of from 50 ppm to 0.05%, both ppm and % referring to ppm by weight or % by weight, respectively, and referring to the total respective inventive solution.

In a one embodiment of the present invention, the concentrated aqueous solutions are free from organic polymers. Free from organic polymers shall mean, in the context of the present invention, that the total contents of organic polymers is 0.1% by weight or less, referring to the amount of inventive mixture. In a preferred embodiment, the term “free from organic polymers” shall encompass a concentration in the range of from 50 ppm to 0.05%, both ppm and % referring to ppm by weight or % by weight, respectively, and referring to the total respective inventive solution. The term “organic polymers” shall also include organic copolymers and shall include polyacrylates, polyethylene imines, and polyvinylpyrolidone. Organic (co)polymers in the context of the present invention shall have a molecular weight (M_{w}) of 1,000 g or more.

In one embodiment of the present invention, the concentrated aqueous solutions do not contain major amounts of alkali metal of mono- and dicarboxylic acids such as acetic acid, propionic acid, maleic acid, acrylic acid, adipic acid, succinic acid, and the like. Major amounts in this context refer to amounts over 0.5% by weight.

In one embodiment of the present invention, aqueous solutions additionally contain at least one inorganic basic salt selected from alkali metal hydroxides and alkali metal carbonates. Examples comprise sodium carbonate, potassium carbonate, potassium hydroxide and in particular sodium hydroxide, for example 0.1 to 1.5% by weight. Potassium hydroxide or sodium hydroxide, respectively, may result from the manufacture of the respective inventive solution,

Furthermore, the mixtures to be used in the present invention as well as aqueous solutions thereof may contain one or more inorganic non-basic salts such as—but not limited to—alkali metal halide or preferably alkali metal sulfate, especially potassium sulfate or even more preferably sodium sulfate. The content of inorganic non-basic salt may be in the range of from 0.10 to 1.5% by weight, referring to the respective inventive mixture or the solids content of the respective inventive solution. Even more preferably, the mixtures to be used in the present invention as well as aqueous solutions thereof do not contain significant amounts of inorganic non-basic salt, for example in the range of from 50 ppm to 0.05% by weight, referring to the respective inventive mixture or the solids content of the respective inventive solution. Even more preferably inventive mixtures contain 1 to 50 ppm by weight of sum of chloride and sulphate, referring to the respective inventive mixture. The contents of sulphate may be determined, for example, by gravimetry or by ion chromatography.

In one embodiment of the present invention the abovementioned mixtures of MGDA with one or more inorganic non-basic salts to be used in the present invention comprise mixtures of L- and D-enantiomers of molecules of general formula (II) as described above, i.e. an enantiomeric mixture of MGDA which is at least partly in the acidic form. Furthermore, inventive mixtures display an improved behaviour towards strong bases such as solid potassium hydroxide or solid sodium hydroxide. When stored as a mixture with solid potassium hydroxide or solid sodium hydroxide and later formulated in water, they can be formulated as clear, non-turbid solutions with good shelve-life.

Method of Manufacturing the MGDA Mixture Used

The mixtures to be used according to the present invention may be prepared by mixing the respective quantities of enantiomerically pure L-MGDA and D-MGDA or their respective salts. However, the manufacture of enantiomerically pure D-MGDA is tedious, and other processes of making the mixtures have been found in the context of the present invention.

In a preferred embodiment the mixtures comprising L-MGDA and D-MGDA may be manufactured by a process comprising at least the steps of
(a) dissolving a mixture of L-alanine and its alkali metal salt in water,
(b) converting said mixture of L-alanine and its alkali metal salt with formaldehyde and hydrocyanic acid or alkali metal cyanide to a dinitrile,
(c) saponification of the dinitrile resulting from step (b) in two steps (c1) and (c2), steps (c1) and (c2) being carried out at different temperatures, employing stoichiometric amounts of hydroxide or an excess of 1.01 to 1.5 moles of hydroxide per molar sum of COOH groups and nitrile groups of dinitrile from step (b).

The process will be described in more detail below,

In step (a) of the process, a mixture of L-alanine and the alkali metal salt of L-alanine is being dissolved in water. L-alanine in the context of the present invention refers to either pure L-alanine with non-detectable amounts of D-alanine, or to mixtures of enantiomers of L-alanine and D-alanine, the enantiomeric excess being at least 96%, preferably at least 98%. The purer the enantiomer L-alanine, the better is the steering of the racemization in step (c) of the inventive process.

Of the alkali metal salts, the potassium salt is preferred and the sodium salt is even more preferred.

There are various ways to perform step (a) of the process. It is possible to prepare a solid mixture of L-alanine and the alkali metal salt of L-alanine and to then dissolve the mixture so obtained in water. It is preferred, though, to slurry L-alanine in water and to then add the required amount alkali metal hydroxide, as solid or as aqueous solution.

In one embodiment, step (a) of the process is being carried out at a temperature in the range of from 5 to 70° C., preferably in the range of from 15 to 60° C. During the performance of step (a), in many instances a raise of temperature can be observed, especially when the embodiment of slurrying L-alanine in water and to then add the required amount alkali metal hydroxide, as solid or as aqueous solution, has been chosen.

An aqueous solution of a mixture of L-alanine and its corresponding alkali metal salt will obtained from step (a).

In one embodiment of step (a), an aqueous solution of a mixture of the range of from 10 to 50 mole-% of L-alanine (free acid) and of 50 to 90 mole-% of L-alanine (alkali metal salt) is being obtained. Particularly preferred are mixtures of 23 to 27 mole-% of L-alanine (free acid) and 63 to 67 mole % of the alkali metal salt of L-alanine is being obtained.

Preferably, an aqueous solution of a mixture of L-alanine and its corresponding alkali metal salt may have a total solids content in the range of from 10 to 35%. Preferably, such aqueous solution of a mixture of L-alanine and its corresponding alkali metal salt may have a pH value in the range of from 6 to 12.

Preferably, the aqueous solution obtained from step (a) contains less than 0.5% by weight, impurities other than D-alanine and its corresponding alkali metal salt, the percentage being based on the total solids content of the aqueous solution. Such potential impurities may be one or more of magnesium or calcium salts of inorganic acids. Trace amounts of impurities stemming from the L-alanine or the water used shall be neglected in the further context with the present invention.

In step (b) of the process, a double Strecker synthesis is being carried out by treating the aqueous solution of the mixture of L-alanine and its alkali metal salt obtained in step (a) with formaldehyde and hydrocyanic acid or alkali metal cyanide. The double Strecker synthesis can be carried out by adding alkali metal cyanide or a mixture from hydrocyanic acid and alkali metal alkali metal cyanide) or preferably hydrocyanic acid and formaldehyde to the aqueous solution obtained in step (a). Said addition of formaldehyde and alkali metal cyanide or preferably hydrocyanic acid can be performed in one or more portions. Formaldehyde can be added as gas or as formalin solution or as paraformaldehyde. Preferred is the addition of formaldehyde as 30 to 35% by weight aqueous solution.

In a particular embodiment of the present invention, step (b) of the inventive process is being carried out at a temperature in the range of from 20 to 80° C., preferably from 35 to 65° C.

In one embodiment, step (b) of the process is being carried out at a constant temperature in the above range. In another embodiment, step (b) of the inventive process is being carried using a temperature profile, for example by starting the reaction at 40° C. and allowing then stirring the reaction mixture at 50° C.

In one embodiment of the present invention, step (b) of the inventive process is being carried out at elevated pressure, for example 1.01 to 6 bar. In another embodiment, step (b) of the inventive process is being carried at normal pressure (1 bar).

In one embodiment, step (b) of the process is being carried out at a constant pH value, and a base or an acid is being added in order to keep the pH value constant. Preferably, however, the pH value during step (b) is decreasing, and neither base nor acid other than, optionally, HCN is being added. In such embodiments, at the end of step (b), the pH value may have dropped to 2 to 4.

Step (b) can be performed in any type of reaction vessel that allows the handling of hydrocyanic acid. Useful are, for example, flasks, stirred tank reactors and cascades of two or more stirred tank reactors.

From step (b), an aqueous solution of the L-enantiomer, a dinitrile of formula (B)

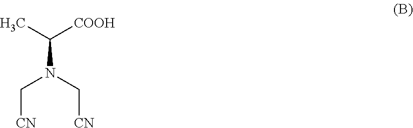

and its corresponding alkali metal salt will be obtained, briefly also referred to as dinitrile (B) or alkali metal salt of dinitrile (B), respectively.

In step (c), the dinitrile resulting from step (b) will be saponified in two steps (c1) and (c2) at different temperatures, employing stoichiometric amounts of hydroxide or an excess of 1.01 to 1.5 moles of hydroxide per molar sum of COOH groups and nitrile groups of dinitrile of step (b), preferably 1.01 to 1.2 moles.

Different temperature means in the context of step (c) that the average temperature of step (c1) is different from the average temperature of step (c2). Preferably, step (c1) is being performed at a temperature lower than step (c2). Even more preferably, step (c2) is being performed at an average temperature that is at least 100° C. higher than the average temperature of step (c1). Hydroxide in the context of step (c) refers to alkali metal hydroxide, preferably potassium hydroxide and even more preferably to sodium hydroxide.

Step (c1) can be started by adding the solution resulting from step (b) to an aqueous solution of alkali metal hydroxide or adding an aqueous solution of alkali metal hydroxide to a solution resulting from step (b). In another embodiment, the solution resulting from step (b) and an aqueous solution of alkali metal hydroxide are being added simultaneously to a vessel.

When calculating the stoichiometric amounts of hydroxide to be added in step (c1), the sum of COOH groups and nitrile groups from the total theoretical amount of dinitrile (B) is multiplied by 3 and the amounts of alkali already present from step (a) and, optionally, step (b), is subtracted.

Step (c1) can be performed at a temperature in the range of from 20 to 80 ° C., preferable 40 to 70° C. In the context of step (c1) “temperature” refers to the average temperature.

As a result of step (c1), an aqueous solution of the respective diamide and its respective alkali metal salt can be obtained, M being alkali metal. Said solution may also contain L-MGDA and the corresponding monoamide and/or its mono- or dialkali metal salt.

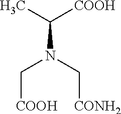

Step (c2) can be performed at a temperature in the range of from 130 to 195° C., preferably 175 to 195° C. In the context of step (c1) “temperature” refers to the average temperature.

In one embodiment, step (c2) has an average residence time in the range of from 5 to 180 minutes.

In preferred embodiments the higher range of the temperature interval of step (c2) such as 190 to 195° C. is combined with a short residence time such as 20 to 25 minutes, or the lower range of the temperature interval of step (c2) such as 175° C. to 180° C. is combined with a longer residence time such as 50 to 60 minutes, or a middle temperature such as 185° C. is combined with a middle residence time such as 35 to 45 minutes.

Step (c2) can be performed in the same reactor as step (c1), or—in the case of a continuous process—in a different reactor.

In one embodiment of the present invention step (c2) is carried out with an excess of base of 1.01 to 1.2 moles of hydroxide per mole of nitrile group.

Depending on the type of reactor in which step (c2) is being performed, such as an ideal plug flow reactor, the average residence time can be replaced by the residence time.

In one embodiment, step (c1) is being carried out in a continuous stirred tank reactor and step (c2) is being carried out in a second continuous stirred tank reactor. In a preferred embodiment, step (c1) is being carried out in a continuous stirred tank reactor and step (c2) is being carried out in a plug flow reactor, such as a tubular reactor.

In one embodiment, step (c1) of the inventive process is being carried out at elevated pressure, for example at 1.05 to 6 bar. In another embodiment, step (c1) of the inventive process is being carried at normal pressure.

Especially in embodiments wherein step (c2) is being carried out in a plug flow reactor, step (c2) may be carried out at elevated pressure such as 1.5 to 40 bar, preferably at least 20 bar. The elevated pressure may be accomplished with the help of a pump or by autogenic pressure elevation.

Preferably, the pressure conditions of steps (c1) and (c2) are combined in the way that step (c2) is carried out at a higher pressure than step (cl).

During step (c2), a partial racemization takes place. Without wishing to be bound by any theory, it is likely that racemization takes place on the stage of of the above L-diamide or of L-MGDA.

In one embodiment, the process may comprise steps other than steps (a), (b) and (c) disclosed above. Such additional steps may be, for example, one or more decolourization steps, for example with activated carbon or with peroxide such as H₂O₂.

A further step other than step (a), (b) or (c) that is preferably carried out after step (c2) is stripping with nitrogen or steam in order to remove ammonia. Said stripping can be carried out at temperatures in the range of from 90 to 110° C. By nitrogen or air stripping, water can be removed from the solution so obtained. Stripping is preferably carried out at a pressure below normal pressure, such as 650 to 950 mbar.

In embodiments wherein an aqueous solution is desired, the solution obtained from step (c2) is just cooled down and, optionally, concentrated by partially removing the water. If dry samples of the mixtures to be used in the present invention are required, the water can be removed by spray drying or spray granulation.

The process described may be carried out as a batch process, or as a semi-continuous or continuous process.

The described process yields a solution of a salt of MGDA, in particular a tri-alkali metal salt or the salt according to formula (I). Naturally, solutions of such salts tri-alkali salts have a basic pH value.

In the processes for recovering crude oil and/or gas from subterranean formations according to the present invention MGDA may be used as basic salt and in basic environment. In other uses less basic or even acidic solutions of MGDA may be used, including but not limited to components of formula (II), i.e. it is necessary to convert the reaction products obtained in step (c) to more acidic products in an additional process step (d).

Techniques for converting salts of MGDA, in particular Na3MGDA to fully or partly acidic forms of MGDA are well known to the skilled artisan and include acidification with acids, ion exchange or electrodialysis. For acidification acids such as sulfuric acid or methane sulfonic acid may be used. Using the acidification technology, some salt such as sodium sulfate or sodium methane sulfonate is also formed as a byproduct and may remain in the product as long as no adverse effects happen when such solutions are used. Converting MGDA salts completely or partly into acidic forms without generating such salts as byproduct may be performed by electrodialysis. Suitable processes are disclosed in WO 2008/065109 A1 and EP 1 004 571 A1.

Use of the MGDA Mixtures for Recovering Crude Oil and/or Gas from Subterranean Formations.

According to the present invention the described mixtures of L- and D-enantiomers of methyl glycine diacetic acid (MGDA) or its respective mono-, di or trialkali metal or mono-, di or triammonium salts containing an excess of the respective L-isomer, wherein the enantiomeric excess (ee) of the L-isomer is in the range of from 10% to 75% are used as additives in processes for the production of crude oil and/or gas from subterranean formations.

The term “recovering of crude oil and/or gas” shall include any processes which may be applied in course of recovering crude oil from subterranean formations starting with drilling wellbores into subterranean formations to processing crude oil and/or gas on the oilfield after producing it from a wellbore. Specifically, the term “recovering of crude oil and/or gas” shall include but be not limited to operations such as drilling of wellbores, completion of wellbores, such as cementing, stimulation such as fracturing and/or acidizing, enhanced oil recovery, conformance control, splitting crude oil—water emulsions, scale inhibition and/or removal of scale form oilfield equipment and/or subterranean formations, iron control, or corrosion protection of oilfield equipment.

In particular, the MGDA mixtures as described above may be used as additives in fluids, in particular aqueous fluids to be used in the abovementioned oilfield operations. Depending on the intended use such fluids may comprise further components including but not limited to surfactants, polymers, such as thickening polymers, bases, acids, or other additives. The concentration of the MGDA mixtures as described above may be selected by the skilled artisan according to the needs of the application and may range from 0.01% by weight to 30% by weight relating to the sum of all components of such aqueous fluid. In one embodiment 0.05% to 2% by weight, preferably 0.1 to 1% by weight the MGDA mixtures as described above are used while in other embodiments higher amounts may be used such as for example 5% by weight to 30% by weight.

For making such aqueous fluids solid MGDA or its respective mono-, di or trialkali metal or mono-, di or triammonium salts may be used and mixed with the fluid, preferably an aqueous and other components. In another embodiment, the abovementioned concentrated aqueous solution of MGDA may be used, mixed with further solvent(s), in particular water and further additives.

Examples of fluids to be used in oilfield operations include drilling fluids, completion fluids, spacer fluids, fracturing fluids, acidizing fluids, fluids for enhanced oil recovery, fluids for conformance control, fluids for iron control or scale removal fluids.

Some preferred oilfield applications are described in the following:

Use of the MGDA Mixtures for Acidizing

In one preferred embodiment of the invention the process in which the described mixtures of L- and D-enantiomers of MGDA and/or salts thereof are used is a process for acidizing subterranean formations. For such acidizing process a preferably aqueous formulation comprising at least the MGDA mixture described above is used.

In course of acidizing subterranean, oil and/or gas bearing formations acids dissolve the rock generating new pores, channels and the like in the formation and/or remove scales present in the formation thereby increasing the permeability of the formation.

In course of acidizing operations iron contaminations may cause problems because the acidic formulations used may dissolve iron and/or iron compounds which are in contact with the acidic formulations. Examples comprise steel equipment such as wellbore tubing or iron containing minerals in the formation which become contacted by the acidic formulation. Such dissolved iron may later, in particular with rising pH value due to the consumption of the acid used, form precipitates with organic or inorganic components—for instance as iron hydroxides thereby plugging the formation or at least hinder the flow of liquids in the formation. Adding MGDA which is able to complex iron (II) and iron (III) ion addresses said problem. Techniques of preventing the precipitation of iron compounds are also known as “iron control”.

In one embodiment of the invention the subterranean formation are selected from formations comprising carbonates. Such subterranean formation may be a subterranean formation which predominantly comprises carbonates, in particular CaCO3 and/or MgCO3, for example in the form of magnesite, dolomite, limestone, chalk or aragonite. Further carbonates, such as, for example, SrCO₃ or BaCO₃, can of course also be present. The subterranean formations can also comprise impurities or the carbonates may be mixed with other rocks, for example silicates. In other embodiments carbonates, in particular CaCO3 and/or MgCO3, may only be present in the formation in minor amounts, preferably in amorphous or poorly crystallized forms. Examples include silicate formations or shale formations comprising some amounts of carbonates, e.g. silicate formations in which silicate and/or quartz particles may be caked together by means of carbonates.

The temperature of the subterranean formations may be from 20° C. to 250° C. Methane sulfonic acid can advantageously be used for the treatment of carbonatic rock formations having a temperature of at least 60° C., in particular from 60 to 250° C.

For carrying out the acidizing method according to the invention, the acidic aqueous formulation is injected into the subterranean formation through at least one wellbore at a pressure sufficient to penetrate into the subterranean formation.

When the acidic aqueous formulation contacts acid-soluble components of a formation, carbonatic components and/or carbonate-containing impurities in the formation react with the acid thereby increasing the permeability of the subterranean formation, By way of example, the increased permeability may be caused by the dissolution of carbonatic impurities clogging pores, cavities and the like in the formation, increasing existing channels, pores and the like and/or forming new channels, pores and the like. The increased permeability yields in a higher oil production when resuming the oil production after the acidizing treatment.

The penetration depth of the acidizing treatment may depend on such parameters as the injection rate, time of treatment but also on the nature of the aqueous formulation itself. When the acid injected is spent than it will have no longer an effect on the formation even if the formulation is forced to penetrate further into the formation.

The acidic aqueous formulation may be injected into a production well or into an injection well. The production well is a well through which mineral oil or natural gas is also withdrawn. The injection well serves for forcing in flooding media for maintaining the pressure in the deposit. A treatment of the injection well reduces pressure drops when the flooding medium is forced in and thus also advantageously contributes to higher productivity.

The acidizing treatment according to the invention can be a so called “matrix acidizing” process. In the case of matrix acidizing the pressure of injection is limited to pressures not sufficient to hydraulically create fissures and/or fractures in the formation.

The acidizing treatment according to the invention may be combined with a fracturing process (the so called “fracture acidizing”). In the case of fracture acidizing the pressure of injection is sufficient to hydraulically create fissures and/or fractures in the formation.

### Acidizing—First Embodiment

In a first embodiment of the acidizing process an aqueous formulation comprising at least water, mixtures of L- and D-enantiomers of MGDA as described above and an acid is used. Optionally, the aqueous formulation may comprise further components including but not limited to surfactants, diverting agents and/or corrosion inhibitors.

Examples of suitable acids comprise HCl, HF, organic acids, such as, for example, formic acid, acetic acid, p-toluenesulfonic acid amido sulfonic acid or water-soluble alkanesulfonic acids. Alkanesulfonic acids have the general formula R¹—SO₃H, where R¹ is a straight-chain, branched or cyclic alkyl radical. In one embodiment of the invention, R¹ is selected from straight-chain or branched C₁- to Cs₆-alkyl, preferably C₁- to Ca₄-alkyl. Most preferably R¹ is methyl, i.e. the acid is methane sulfonic acid.

Of course also a mixture of two or more acids may be used. Examples of suitable acid mixtures comprise mixtures of methanesulfonic acid and HF, methanesulfonic acid and HCl, formic acid and acetic acid, acetic acid and HCl, formic acid and HCl, and HF and HCl. Mixtures of HF and HCl are also known as mud acid any may be used for example in a weight ratio of 9:1 or 12:3.

The skilled artisan may select suitable acids according to his/her needs, in particular according to the nature of the formation to be acidized. By way of example if the formation comprises silicates and it is desired also to dissolve such silicates HF or acid mixtures comprising HF should be used.

Methanesulfonic acid (abbreviated to MSA, formula: H₃C—SO₃H) is particularly preferably used. Methanesulfonic acid is a very strong acid (pKₐ:−2) but, in contrast to HCl or formic acid, has only a low vapor pressure. It is therefore very particularly suitable also for use at relatively high temperatures. Methanesulfonic acid can therefore advantageously be used for the treatment of subterranean formations having a temperature of at least 60° C., in particular from 60 to 250° C.

The concentration of the acids in the aqueous solutions may be chosen by the skilled artisan according to his/her needs. It is self-evident that it depends on the nature of the acid.

By way of example the concentration of methanesulfonic acid may be from 1% to 50% by weight with respect to all components of the aqueous solution, in particular from 5% to 50% by weight, preferably 10% to 30% by weight, and for example 15% to 25% by weight.

The concentration of HCl used may be from 2% to 28% by weight in particular 2 to 20% by weight, preferably 5% to 15% by weight.

Besides water and an acid, the aqueous formulation used may comprise small amounts of organic, water-miscible solvents. These may be, in particular, alcohols, for example methanol, ethanol or propanol, however as a rule, the proportion of water is at least 80% by weight, preferably 90% by weight and particularly preferably at least 95% by weight, based in each case on the total amount of all solvents used.

The amount of MGDA and/or its respective salts as described above in the aqueous formulation of this first embodiment are selected by the skilled artisan according to his/her needs. In particular, the concentration may be from 0.05% to 2% by weight, preferably 0.1 to 1% by weight, relating to the total of all components of the aqueous formulation.

The pH-value of the aqueous formulation in particular depends on the nature and amount of the acid used. It may be from 0 to 5, for example from 0 to 2.

The aqueous formulation comprising MGDA as described above can of course also comprise conventional additives and assistants which are typical for an acidizing treatment of oil- or gas-carrying rock formations. Examples of such assistants comprise, for example, polymers for increasing the viscosity, foam formers or foam breakers, oxidizing agents, enzymes, assistants for reducing the friction or for controlling paraffin precipitations and biocides or surfactants, and in particular retarding surfactants.

In addition to MGDA also further complexing agents and/or their respective salts may be used, including but not limited to glutamic acid N,N-diacetic acid (GLDA), nitrilotriacetic acid (NTA), ethylenediaminetetraacetic acid (EDTA), diethylenetriaminepentaacetic acid (DTPA), or hydroxyethylethylenediaminetriacetic acid (HEDTA). The content of additional additives is chosen by the person skilled in the art according to the desired use.

In one embodiment of the invention the aqueous formulation used according to the present invention comprises at least water, an acid, the described MGDA mixture comprising an excess of the L-enantiomer and a corrosion inhibitor soluble in the acidic aqueous formulation.

Examples of suitable corrosion inhibitors are known and may be selected by the skilled artisan according to his/her needs. Of course, mixtures of different corrosion inhibitors may also be used. The content of corrosion inhibitors is chosen by the person skilled in the art according to the desired use.

Examples of suitable water-soluble corrosion inhibitors comprise alkyne derivatives, for example propargyl alcohol or 1,4-butynediol.

In a preferred embodiment of the invention, said derivatives are alkoxylated alkyne derivatives of the general formula

HC≡C—CH₂—O(—CH₂—CHR²—O—)ₙH (III), or

H(—O—CHR²—CH₂—)ₙ—O—CH₂—C≡C—CH₂—O(—CH₂—CHR²—O—)_{n′}H (IV),

where the radicals R², in each case independently of one another, are H or methyl and the indices n and n′, independently of one another, are from 1 to 10.

It is known to the person skilled in the art that such alkoxy groups are obtainable in particular by oxyalkylation or starting from industrial polyglycols. Said values for n are thus average chain lengths, and the average value need not of course be a natural number but may also be any desired rational number n and n′ are preferably a number from 1 to 3. The alkyleneoxy groups may be exclusively groups derived from ethylene oxide units or exclusively groups derived from propylene oxide. However, they may of course also be groups which have both ethylene oxide units and propylene oxide units. Polyoxyethylene units are preferred.

In one further embodiment of the invention the aqueous formulation used according to the present invention comprises at least water, an acid, the described MGDA mixture comprising an excess of the L-enantiomer, and a surfactant. In a preferred embodiment, the formulation comprises additionally a corrosion inhibitor soluble in the acidic aqueous formulation as described above.

Suitable surfactants are known to the skilled artisan and may be selected according to his/her needs and may be selected from the group of anionic surfactants, cationic surfactants, non-ionic surfactants, amphoteric surfactants or zwitterionic surfactants. Of course mixtures of two or more surfactants may be selected. Examples of suitable surfactants comprise alk(en)nylpoly-glucosides, alkylpolyalkoxylates, alkylphenolalkoxylates, sorbitan esters which may optionally be alkoxylated, alkanolamides, amine oxides, alkoxylated fatty acids, alkoxylated fatty amines alkoxylated alkyl amines or quaternary ammonium compounds.

Surfactants may serve several functions in formulations for acidizing.

In one embodiment, foaming surfactants may be used. Formulations for acidizing comprising such foaming surfactants may be foamed by mixing the formulations with a gas before or during injection into the subterranean formation. Examples of gases include nitrogen, carbon dioxide or methane. Preferably, the gas is nitrogen. Foaming surfactants stabilize the gas-liquid interface. Foams of acidizing formulations have a higher viscosity than liquid formulations and therefore flow more uniformly into the subterranean formation. Examples of suitable foaming surfactants comprise alkyl sulfates such as sodium lauryl sulfate, betaines such as alkylamidobetaines, amine oxides, or quaternary ammonium compounds such as trimethyl tallow ammonium salts.

In another embodiment, the surfactants are so called retarding surfactants. If very reactive acids such as HCl are used for acidizing the acids quickly reacts with the formation once it gets into contact with the formation and therefore the acid may quickly become spent in the near wellbore regions of the respective subterranean formation. Retarding surfactants slow down the reaction between the acid and the formation thereby allowing the acid to penetrate deeper into the formation. In one embodiment so called “wormholes” may be formed. Examples of suitable retarding surfactants comprise sulfonates of the general formula RSO₃X, wherein R is a C₈ to C₂₅ hydrocarbon moiety and X is an alkali metal ion or an ammonium ion.

In another embodiment an emulsion of the described aqueous acidic formulations in non-polar organic solvents may be used. Examples of suitable organic solvents comprise hydrocarbons such as xylene or toluene or high boiling (e.g. having a boiling point of at least 160° C. at normal pressure) aromatic and/or aliphatic hydrocarbon fractions. Preferably, a surfactant for stabilizing the emulsion should be used. Such emulsions may also be used for retarding the action of the acid on the formation thereby allowing the acid to penetrate deeper into the formation.

In one embodiment the acidic aqueous formulation is specifically adapted for fracture acidizing and comprises at least water, the MGDA mixture described above, a proppant and thickening components such as thickening surfactants and/or thickening polymers. Proppants are known to the skilled artisan. They are small hard inorganic or organic particles, for instance sand particles which are transported by the fluid into newly formed fractures or fissions in order to keep them open after pressure has been released. Examples of thickening polymers comprise polyacrylamide or copolymers of acryl amide and other water soluble monomers such as for instance acrylic acid.

### Acidizing—Second Embodiment

In a second embodiment of the acidizing process an aqueous formulation comprising at least water and at least mixtures of L- and D-enantiomers of MGDA molecules of the general formula (II) as defined above are used, preferably molecules for which y is from 1 to 2.5, more preferably from 1.5 to 2.5.

An additional acid may optionally be present but is not required, i.e. in this second embodiment an aqueous solution comprising L- and D-enantiomers of molecules of the general formula (II) is used as the acidic component for acidizing the formation.

The concentration of MGDA molecules of the general formula (II) in this second embodiment is selected by the skilled artisan according to his/her needs and may in particular be from 2.5% to 30% by weight with respect to the total of all components of the formulation used, preferably 5% to 25% by weight and for example 10% to 20% by weight.

Preferred aqueous formulations have a pH value of from 3 to 6.

Besides the mixture of L- and D-enantiomers of MGDA molecules of the general formula (II) the aqueous formulation for acidizing may comprise conventional additives and assistants which are typical for an acidizing treatment of oil- or gas-carrying rock formations as already described above.

Examples of further components have already been described above for the first embodiment of acidizing and we refer to said description.

Use of the MGDA Mixtures for Iron Control

In another preferred embodiment of the invention the process the process in which the MGDA mixtures described above and/or salts thereof are used is a process of controlling iron.

In course of using acidic formulations in processes for recovering crude oil and/or gas from subterranean formations the formulations may contact steel equipment such as wellbore tubing, and or iron compounds in the formation or at the surfaces of equipment such as rust and the acidic formulation used may dissolve iron and/or iron compounds which are in contact with the acidic formulation. Furthermore, used components of aqueous formulations such as for instance acids may comprise iron impurities. Such dissolved iron may later, in particular with rising pH value due to the consumption of acid, form precipitates with organic or inorganic components—for instance as iron hydroxides thereby plugging the formation or at least hinder the flow of liquids in the formation and/or equipment.

Adding MGDA which is able to complex iron (II) and iron (III) ion addresses said problem. Techniques of preventing the precipitation of iron compounds are also known as “iron control”.

For iron control the MGDA mixtures described above, including but not limited to the mixtures comprising molecules of formula (II) may be added to aqueous formulations for oilfield uses.

The amount of MGDA and/or its respective salts as described above in such aqueous formulations is selected by the skilled artisan according to his/her needs. In particular, the concentration may be from 0.05% to 2% by weight, preferably 0.1 to 1% by weight, relating to the total of all components of the aqueous formulation.

Use of the MGDA Mixtures for the Inhibition and/or Dissolution of Scale

In another preferred embodiment of the invention the process in which the MGDA mixtures described above and/or salts thereof are used is a process for the inhibition and/or dissolution of scale. Components for the inhibition and/or dissolution of scale are often simply termed as “scale inhibitors”.

In the course of mineral oil and/or natural gas production, solid deposits of inorganic or organic substances can form in a mineral oil and/or natural gas containing subterranean formation itself, in underground installation parts, for example the well lined with metal tubes, and in above-ground installation parts, for example separators. The formation of such solid deposits is extremely undesirable because they can at least hinder the production of mineral oil or natural gas and, in the extreme case, lead to complete blockage of the installation parts affected. Such deposited scales typically comprise carbonates such as calcium carbonate or magnesium carbonate but also sulfates, such as calcium sulfate, strontium sulfate or barium sulfate.

Such scales may be dissolved using suitable aqueous formulations comprising at least water and the MGDA mixture described above. In one embodiment an aqueous formulations comprising at least water and the MGDA mixture comprising molecules of formula (II) as described above may be used.

The amount of MGDA and/or its respective salts as described above in such aqueous formulations is selected by the skilled artisan according to his/her needs. In particular, the concentration may be from 0.5% to 10% by weight, preferably 1 to 3% by weight, relating to the total of all components of the aqueous formulation.

The aqueous formulation for scale inhibition may optionally comprise further components, in particular acids and/or further scale inhibitors, so-called co-inhibitors.

Examples of acids comprise HCl and in particular methane sulfonic acid.

Examples of co-inhibitors include complexing agents other than MGDA and/or their respective salts, including but not limited to glutamic acid N,N-diacetic acid (GLDA), nitrilotriacetic acid (NTA), ethylenediaminetetraacetic acid (EDTA), diethylenetriaminepentaacetic acid (DTPA), or hydroxyethylethylenediaminetriacetic acid (HEDTA). Further examples comprise polyacrylic acid or copolymers comprising acrylic acid or salts thereof, phosphate or phosphonate groups containing compounds such as alkylphenolphosphate ester acids, hydroxy-amino phosphates esters, polymers comprising phosphate or phosphonate groups.

By way of example, the weight ratio of inventive mixture to co-inhibitor may be in the range of from 10:1 to 1:10.

For performing the process of scale inhibition the surfaces with deposited scales are brought into contact with the aqueous formulation described, for example by rinsing installations with the aqueous formulation.

Use of the MGDA Mixtures for the Removal of Filter Cakes

In another preferred embodiment of the invention the process in which the MGDA mixtures described above and/or salts thereof are used is a process of removing filter cakes.

In oil well operations a filter cake may be formed by the introduction of a material capable of forming an impermeable layer on the walls of the wellbore, in particular during the drilling. Such a filter cake prevents the flow of drilling fluid into the subterranean formation. Let aside the loss of drilling fluid, drilling fluid in the formation may damage the near wellbore area and thereby hindering the flow of oil or gas from the formation into the wellbore. For this reason such a filter cake has to be removed.

For the process of removing filter cakes according to the present invention suitable aqueous formulations comprising at least water and the MGDA mixture described above are used. In one embodiment an aqueous formulations comprising at least water and the MGDA mixture comprising molecules of formula (II) as described above may be used.

The amount of MGDA and/or its respective salts as described above in such aqueous formulations is selected by the skilled artisan according to his/her needs. In particular, the concentration may be from 0.1% to 40% by weight, preferably 5 to 20% by weight, relating to the total of all components of the aqueous formulation.

Suitable formulations for removing filter cakes may of course optionally comprise further components such as those disclosed in U.S. Pat. No. 6,494,263 B2.

For performing the process of removing filter cakes the filter cake is brought into contact with the aqueous formulation described by injecting the aqueous formulation into the wellbore.

Use of the MGDA Mixtures for Enhanced Oil Recovery.

In another preferred embodiment of the invention the process in which the MGDA mixtures described above and/or salts thereof are used is a process of enhanced oil recovery, preferably a process of alkali-surfactant or alkali-surfactant-polymer flooding (the latter one also known as “ASP flooding”).

In enhanced oil recovery operations an aqueous formulation comprising at least one surfactant and/or a thickening, water-soluble polymer is injected into a mineral oil deposit through at least one injection well and crude oil is withdrawn from the deposit through at least one production well. Water-soluble polymers and/or surfactants mobilize oil which otherwise remained caught in the subterranean formation.

Crude oil usually comprises acids such as naphtenic acids. Adding bases to aqueous formulations for enhanced oil recovery, i.e. using aqueous fluids having a pH-value of more than 7, preferably 9 to 13 results—after injection into the formation- in a conversion of said naturally occuring acids into the respective salts. Such salts of naphtenic acids have surface active properties and support the process of mobilizing oil.

Besides crude oil subterranean oil bearing formations comprise formation water which usually comprises salts, including but not limited to bivalent ions such as Ca²⁺ or Mg²⁺. Often formation water is also used as fluid for making the aqueous formulations for enhanced oil recovery. If said Ca²⁺ and/or Mg²⁺ ions come into contact with bases hydroxides may precipitate and plug the formation.

For the process of enhanced oil recovery according to the present invention an aqueous solution is used which comprises at least water, a base, a surfactant and the MGDA mixtures described above and/or salts thereof.

Preferably, the MGDA mixtures comprising molecules of formula (I) as described above, more preferably tri alkali or tri ammonium salts of the MGDA mixture described above are used.

The amount of MGDA and/or its respective salts as described above in such aqueous formulations is selected by the skilled artisan according to his/her needs. In particular, the concentration may be from 0.1% to 40% by weight, preferably 0.2 to 20% by weight, for example 0.5 to 2% by weight relating to the total of all components of the aqueous formulation.

The water to be used for the aqueous formulation may be fresh water, sea water, brine or formation water or mixtures thereof.

In principle, it is possible to use any kind of base with which the desired pH can be attained, and the person skilled in the art will make a suitable selection. Examples of suitable bases include alkali metal hydroxides, for example NaOH or KOH, or alkali metal carbonates, for example Na₂CO₃.

The pH of the aqueous formulation is generally at least 8, preferably at least 9, especially 9 to 13, preferably 10 to 12 and, for example, 10.5 to 11.

Examples of suitable surfactants for surfactant flooding include surfactants comprising sulfate groups, sulfonate groups, polyoxyalkylene groups, anionically modified polyoxyalkylene groups, betaine groups, glucoside groups or amine oxide groups, for example alkylbenzenesulfonates, olefinsulfonates, amidopropyl betaines, alkyl polyglucosides, alkyl polyalkoxylates or alkyl poly-alkoxysulfates, -sulfonates or -carboxylates. It is possible with preference to use anionic surfactants, optionally in combination with nonionic surfactants. The concentration of the surfactants is generally 0.01% by weight to 2% by weight, preferably 0.05 to 1% by weight and, for example, 0.1 to 0.8% by weight, based on the sum total of all the components of the aqueous formulation.

Optionally, the formulation used may comprise at least one water soluble, thickening polymer. Examples of thickening polymers for enhanced oil recovery comprise high molecular weight polyacrylamides or copolymers of acryl amide such as acrylamide-acrylic acid copolymers. Usually, such polymers have a weight average molecular weight of at least 500,000 g/mol, preferably 1 to 30 Mio g/mol.

Optionally, the aqueous formulations may of course comprise further components. Examples of further components include biocides, stabilizers, free-radical scavengers, inhibitors or cosolvents.

For performing the process of enhanced oil recovery the formulations described above are injected into a mineral oil deposit through at least one injection well and crude oil is withdrawn from the deposit through at least one production well.

The Invention is Further Illustrated by Working Examples

General Remarks:

The ee value was determined by HPLC using a Chirex 3126 column; (D)-penicillamine, 5 μm, 250×4.6mm. The mobile phase (eluent) was 0.5 mM aqueous CuSO₄-solution. Injection: 10 μl, flow: 1.5 ml/min. Detection by UV light at 254 nm. Temperature: 20° C. Running time was 25 min. The ee value was determined as difference of the area% of the L- and D-MGDA peak divided by the sum of area% of L- and D-MGDA peak. Sample preparation: A 10 ml measuring flask was charged with 5 mg of test material and then filled mark with the eluent and then homogenized.

In each case, the solubility was calculated to refer to pure MGDA, without hydrate water.

I. Syntheses of the MGDA Mixtures

With exception of ee values, percentages in the context of the examples refer to percent by weight unless expressly indicated otherwise.

I.1 Synthesis of a Solution of Partially Neutralized of L-alanine, Step (a.1)

A 5-litre stirred flask was charged with 2,100 g of de-ionized water and heated to 40° C. 1,200 g of L-alanine (13.47 mol, 98% ee) were added. To the resultant slurry 700 g of 50% by weight aqueous sodium hydroxide solution (8.75 mol) were added over a period of 30 minutes. During the addition the temperature raised to 60° C. After complete addition of the sodium hydroxide the slurry was stirred at 60° for 30 minutes. A clear solution was obtained.

Samples of aqueous L-MGDA-Na3 were prepared according to WO 2012/150155, page 4, lines 26 ff.

I.2 Synthesis of Aqueous Solutions of L-MGDA-Na₃, Steps (b.1) and Saponification

A 5-litre stirred flask was charged with 500 ml of water and heated to 40° C. Then, 2,373 g of L-alanine solution according to step (a.1) (8.00 mole), 1627 g of 30% by weight aqueous formaldehyde solution (16.27 mole) and 220 g of hydrogen cyanide (8.15 mol) were added simultaneously within 60 minutes. Then, additional 220 g of hydrogen cyanide (8.15 mol) were added at 40 ° C. within 60 minutes. Upon completion of the addition the reaction mixture was stirred for additional 60 minutes at 40 ° C. The resulting solution of dinitrile (B) and 1,600 g of sodium hydroxide (50% aqueous solution, 20 mol) were simultaneously added over 160 minutes into another 5-litre stirred flask at a constant rate. During that addition, the temperature was maintained at 28 to 32 ° C. by external cooling. Upon completion of the addition, the reaction mixture was refluxed for 6 hours at 90 to 95 ° C. to complete the saponification and to remove any excess of ammonia. The resultant reaction mixture was treated with activated carbon. The resulting aqueous solution contained 38.00 wt % L-MGDA-Na3 and 0.08 wt % trisodium nitrilotriacetate (NTA-Na₃). The enantiomeric excess of L-MGDA-Na₃ (94.3%) was determined by the aforementioned HPLC method.

The above aqueous solution of MGDA was used as starting material to investigate the racemization behavior of L-MGDA-Na₃: 6 g of the solution were filled into a pressure tube. This tube was sealed and then tempered for a defined period of time (table 1) at 120° C. or 180° C., respectively. Then the solution was cooled to room temperature and the enantiomeric excess was determined by HPLC.

TABLE 1
Heating of L-MGDA-Na₃ (6 g of 38% aqueous
solution, excess of 0.15 mol NaOH/mol
MGDA) in a sealed pressure tube.
C-0 and C-(c2.1) are comparative examples.

| | Temperature | Period | L-MGDA-Na₃ | D-MGDA-Na₃ | ee |
|---|---|---|---|---|---|
| # | [° C.] | [min] | [%] | [%] | [%] |
| C0 | — | — | 97.1 | 2.9 | 94.3 |
| C (c2.1) | 120 | 60 | 94.4 | 5.6 | 88.8 |
| (c2.2) | 180 | 20 | 78.3 | 21.7 | 56.6 |
| (c2.3) | 180 | 40 | 61.3 | 38.7 | 22.6 |
| (c2.4) | 180 | 60 | 55.8 | 44.2 | 11.6 |

The mixtures so obtained typically have an NTA-Na₃ content of 0.05% by weight with respect to the total solution. Unlike the mixture obtained from C-(c2.1), inventive mixtures displayed an excellent olfactory behavior, and they had a low tendency of yellowing.

I.2 Synthesis of Aqueous Solutions of Inventive Mixtures, Steps (b1) and (c1.1) and (c2.5) to (c2.7), continuous process

The continuous syntheses of ca. 40% solutions of inventive mixtures were carried out in cascade of 6 stirred tank reactors, total volume of 8.5 I. The reaction mixture passed all 6 stirred tank reactors (STR.1 to STR.6) consecutively. The last stirred tank reactor to be passed, STR.6, was connected to a tubular reactor, TR.7. In the first three stirred tank reactors, STR.1 to STR.3, dinitrile (B) was synthesized, and STR.1 to STR.3 were operated at 40° C. The average residence time in STR.1 to STR.3 was 45 to 90 min in total. In the three stirred tank reactors STR.4 to STR.6 the saponification was carried out. STR.4 to STR.6 were operated at 60° C. The average residence time in STR.4 to STR.6 was 170 to 400 min in total. The saponification was then completed in tubular reactor TR.7 which was operated with a temperature profile of 130 to 195° C. The final ammonia stripping is done in a column under normal pressure using steam. Formaldehyde (30% aqueous solution), an aqueous solution of L-alanine (I) and its sodium salt obtained according to 1.1 and 80 mol-% of the required HCN were added to STR.1, the remaining 20% of the required HCN were added to STR.2, the required sodium hydroxide solution was added in STR.4.

The molar ratios of the feed materials were as follows:

Sum of L-alanine and its sodium salt: 1.00,

Formaldehyde=1.95 to 2.05,

HCN=1.95 to 2.10 and

Sodium hydroxide=3.15 (total amount of sodium hydroxide, including sodium hydroxide added in step (a.1).

The 40% by weight solutions of inventive mixtures so obtained typically had an NTA-Na₃ content of 0.05-0.10% with respect to the total respective solution. They displayed excellent olfactory behavior, and they had a low tendency of yellowing.

TABLE 2
Influence of temperature and residence time of step (c2.5) to (c2.7)

| | Temperature [° C.] | Residence time for step (c2) [min] | ee [%] |
|---|---|---|---|
| (c2.5) | 180 | 70 | 10.0 |
| (c2.6) | 180 | 30 | 30.6 |
| (c2.7) | 178 | 30 | 36.2 |

II. Application Tests

In order to evaluate the inventive mixture with respect to its handling for oilfield application the solubility of the mixture in water was analyzed and furthermore the stability of aqueous solutions against strong alkali was analyzed. Stability against alkali is important for oilfield applications which use alkaline formulations such as alkali-surfactant-flooding or alkali-surfactant-polymer-flooding.

II. 1 Determination of Solubility in Water

The solubility of inventive mixtures and of comparison experiments was determined with an apparatus that allowed heating and cooling down liquid solutions. The temperature (or concentration) of beginning solubility was determined by first cooling down a 50% by weight solution of the respective inventive mixture (or of pure L-MGDA, or of racemic MGDA) to beginning precipitation of MGDA, cooling rate: 30° C./h. Then, the slurry of crystals of MGDA so obtained was again heated at a speed of 1° C./h until a clear solution was obtained. The slurry/solution was checked for solid particles by visible light scattering detection.

The above tests furnished the minimum temperature, Tₛₒₗ, for forming a kinetically stable 50% by weight solution of the respective mixture of isomers. A higher temperature Tₛₒₗ correlates with a lower solubility at ambient temperature.

TABLE 3
Correlation ee value versus Tₛₒₗ

| ee value (%) | Tₛₒₗ [° C.] |
|---|---|
| zero | 103.8 |
| 10.1 | 74.5 |
| 20.0 | 74.0 |
| 50.0 | 68.1 |
| 70.0 | 59.5 |
| 97.7 | 37.0 |

The examples show that for the racemic mixture (ee value 0%) a temperature of 103.8° C. was necessary to obtain a clear solution having a concentration of 50% by weight of MGDA. With increasing ee-values the temperature for obtaining a clear a clear solution having a concentration of 50% by weight of MGDA becomes lower and lower, i.e. its solubility becomes better.

II.2 Determination of Stability Against Strong Alkali

25 g of a 40% by weight aqueous solution of the respective inventive mixture (or of racemic MGDA) were mixed under one hour of stirring with the amount of solid NaOH beads according to table 5. The mixture so obtained was stored at ambient temperature over the period of up to 96 hours. Then, the appearance was characterized by visual inspection. The results are summarized in table 4.

TABLE 4
Appearance of test solutions after 96 h of storage

| NaOH added | | | |
|---|---|---|---|
| [g] | [wt. % vs. 40% solution] | Racemic MGDA | MGDA: 50% ee |
| 0 | 0 | clear liquid | clear liquid |
| 0.5 | 2 | clear liquid | clear liquid |
| 1.0 | 4 | clear liquid | clear liquid |
| 1.5 | 6 | turbid* liquid | clear liquid |
| 2.0 | 8 | turbid* liquid | clear liquid |
| 2.5 | 10 | turbid, precipitate** | clear liquid |

*after 48 hours
**immediately after addition of NaOH

The examples show that the solubility of the racemic MGDA decreases with increasing NaOH amounts while the mixture according to the present invention (50% ee) remains clear.

1.-23. (canceled)

24. A process for recovering crude oil and/or gas from subterranean formations which comprises adding methyl glycine diatetic acid (MGDA) as additive to the subterranean formulations, wherein the MGDA is a mixture of L- and D-enantiomers of MGDA or its respective mono-, di or trialkali metal or mono-, di- or triammonium salts, said mixture containing an excess of the respective L-isomer, wherein the enantiomeric excess (ee) of the L-isomer is in the range of from 10% to 75%.

25. The process according to claim 24, wherein the process is selected from the group consisting of drilling, completion of wellbores, stimulation, enhanced oil recovery, conformance control, splitting of crude oil-water emulsions, scale inhibition and; or removal of scale form oilfield equipment and/or subterranean formations, iron control, and corrosion protection of oilfield equipment.

26. The process according to claim 24, wherein the process is a process for acidizing subterranean formations comprising at least injecting an aqueous formulation comprising at least water, the MGDA mixture described in claim 24 and an acid into a subterranean formation through at least one wellbore at a pressure sufficient to penetrate said subterranean formation.

27. The process according to claim 26, wherein the formation comprises carbonates.

28. The process according to claim 26, wherein the acid is at least one selected from the group of HCl, HF, formic acid, acetic acid, p-toluenesulfonic acid amido sulfonic acid and water-soluble alkanesulfonic acids having the general formula R¹—SO₃H, wherein R¹ is an C₁- to C₄-alkyl moiety.

29. The process according to claim 26, wherein the acid is methane sulfonic acid.

30. The process according to claim 29, wherein the concentration of the methane sulfonic acid is from 1% to 50% by weight with respect to all components of the aqueous formulation.

31. The process according to claim 26, wherein the concentration of the MGDA mixture is from 0.05% to 2% by weight relating to the total of all components of the aqueous formulation.

32. The process according to claim 26, wherein the aqueous formulation additionally comprises at least one corrosion inhibitor.

33. The process according to claim 26, wherein the aqueous formulation additionally comprises at least one surfactant.

34. The process according to claim 33, wherein the surfactant is a retarding surfactant.

35. The process according to claim 33, wherein the surfactant is a foaming surfactant and the aqueous formulation is foamed by mixing it with a gas before or during injection into the formation.

36. The process according to claim 35, wherein the gas is selected from the group of nitrogen, carbon dioxide and methane.

37. The process according to claim 26, wherein an emulsion of the aqueous formulation in non-polar organic solvents is used.

38. A mixture of L and D-enantiomers of molecules of general formula (II)
[CH₃—CH(COO)—N(CH₂—COO)₂]M_{3-y}H_{y} (II),
wherein y is in the range of from 0.75 to 2.9, M is selected from substituted or non-substituted ammonium, potassium and sodium or mixtures thereof, said mixture containing an excess of the respective L-isomer, wherein the enantiomeric excess (ee) is in the range of from 10 to 75%.

39. The mixture according to claim 38, wherein y is in the range from 1 to 2.5 and ee is in the range of from 12.5 to 60%.

40. A process for acidizing subterranean formations comprising at least injecting an aqueous formulation comprising at least water and an MGDA mixture according to claim 38 into the subterranean formation through at least one wellbore at a pressure sufficient to penetrate said subterranean formation.

41. The process according to claim 40, wherein the concentration of the MGDA mixture is from 2.5% to 30% by weight relating to the total of all components of the aqueous formulation.

42. The process according to claim 24, wherein the process is a process for iron control.

43. The process according to claim 24, wherein the process is a process for the inhibition and/or dissolution of scale.

44. The process according to claim 24, wherein the process is a process for removing filter cakes.

45. The process according to claim 24, wherein the process is a process of enhanced oil recovery comprising at least injecting an aqueous formulation comprising at least water, a base, a surfactant and an MGDA mixtures described in claim 24 into the subterranean, oil bearing formation through at least one injection well and withdrawing crude oil from the formation through at least one production well.

46. The process according to claim 45, wherein the aqueous formulation additionally comprises at least one water soluble thickening polymer.
